(19) United States
(12) Patent Application Publication
(10) Pub. No.: US 2025/0025288 A1
(43) Pub. Date: Jan. 23, 2025

(54) GRAFT AND SCAFFOLD ACL REPAIR SYSTEM

(71) Applicant: Miach Orthopaedics, Inc., Westborough, MA (US)

(72) Inventors: Rita PAPARAZZO, Hopkinton, MA (US); Stephen WOHLERT, Flemington, NJ (US)

(73) Assignee: Miach Orthopaedics, Inc., Westborough, MA (US)

(21) Appl. No.: 18/777,010

(22) Filed: Jul. 18, 2024

Related U.S. Application Data
(60) Provisional application No. 63/527,655, filed on Jul. 19, 2023.

(51) Int. Cl. A61F 2/08; A61L 27/24; A61L 27/36

(52) U.S. Cl. CPC A61F 2/0811; A61L 27/24; A61L 27/3662; A61L 2430/10

(57) ABSTRACT

An arthroscopic system for the repair of a ruptured ligament is provided. Aspects of the invention include a scaffold surrounding a graft and attached by a suture to an fixation device and inserted into a repair site via arthroscopic equipment. The scaffold, the graft, and suture are further secured near or at the repair site via arthroscopic equipment having an elongated delivery member that contains the scaffold, graft, and suture prior to inserting the scaffold, graft, and suture into the repair site.

### CROSS-REFERENCE TO RELATED APPLICATIONS

The present application claims the benefit of and priority to U.S. Provisional Application No. 63/527,655, filed Jul. 19, 2023, the entire contents of which are incorporated by reference into this application.

### FIELD OF THE INVENTION

The invention relates generally to systems and methods for the repair of a ruptured ligament utilizing an arthroscopic repair system having a combination of a scaffold implant encasing a graft.

### BACKGROUND OF THE INVENTION

Intra-articular tissues, such as the anterior cruciate ligament (ACL), do not heal after rupture. In addition, the meniscus and the articular cartilage in human joints also often fail to heal after an injury. Tissues found outside of joints heal by forming a fibrin clot, which connects the ruptured tissue ends and is subsequently remodeled to form a scar, which heals the tissue. Inside a synovial joint, a fibrin clot either fails to form or is quickly lysed after injury to the knee, thus preventing joint arthrosis and stiffness after minor injury. Joints contain synovial fluid which, as part of normal joint activity, naturally prevent clot formation in joints. This fibrinolytic process results in premature loss of the fibrin clot scaffold and disruption of the healing process for tissues within the joint or within intra-articular tissues.

The current treatment method for human anterior cruciate ligament repair after rupture involves removing the ruptured fan-shaped ligament and replacing it with a point-to-point tendon graft (ACL reconstruction). While this procedure can initially restore gross stability in most patients, longer follow-up demonstrates many post-operative patients have abnormal structural laxity, suggesting the reconstruction may not withstand the physiologic forces applied over time (Dye, 325 Clin. Orthop. 130-139 (1996)). The loss of anterior cruciate ligament function has been found to result in early and progressive radiographic changes consistent with joint deterioration (Hefti et al., 73A (3) J. Bone Joint Surg. 373-383 (1991)), and over 70% of patients undergoing ACL reconstruction develop osteoarthritis at only 14 years after injury (von Porat et al., Ann Rheum Dis. 63 (3): 269-73 (2004)). As anterior cruciate ligament rupture is most commonly an injury of young athletes in their teens and twenties, early osteoarthritis in this group has difficult consequences.

### SUMMARY OF THE INVENTION

An embodiment of the present disclosure includes a ligament repair system. The ligament repair system includes an implant sized and shaped for placement in synovial fluid, wherein the implant is configured to absorb a repair material. The ligament repair system further includes one or more suture assemblies configured to position the implant along or adjacent to a ruptured end of a torn ligament. The ligament repair system further includes a first fixation device configured to couple the one or more suture assemblies to a first bone. The ligament repair system further includes a tendon graft configured to be placed adjacent to the implant. The ligament repair system further includes at least one graft suture configured to position the graft along or adjacent to the implant or the ruptured end of the torn ligament. The ligament repair system further includes a second fixation device configured to couple the at least one graft suture to the first bone.

A further embodiment of the present disclosure includes a method for repairing an anterior cruciate ligament. The method includes inserting a scaffold proximate a ruptured end of a torn ligament. The method includes securing at least one scaffold to the scaffold and a first bone with a first fixation device. The method includes coupling a graft to the scaffold. The method includes positioning the graft along the scaffold or the ruptured end of the torn ligament via at least one graft suture. The method further includes coupling the scaffold to the a second bone with a second fixation device.

A further embodiment of the present disclosure includes a ligament repair system. The ligament repair system includes an implant sized and shaped for placement in synovial fluid, wherein the implant is configured to absorb a repair material. The ligament repair system further includes one or more suture assemblies configured to position the implant along or adjacent to a ruptured end of a torn ligament. The ligament repair system further includes a first fixation device configured to couple the one or more suture assemblies to a first bone. The ligament repair system further includes an autologous tendon graft configured to be placed adjacent to the implant. The ligament repair system further includes at least one graft suture configured to position the graft along or adjacent to the implant or the ruptured end of the torn ligament. The ligament repair system further includes a second fixation device configured to couple the at least one graft suture to the first bone.

A further embodiment of the present disclosure includes a ligament repair system. The ligament repair system includes an implant sized and shaped for placement in synovial fluid, wherein the implant is configured to absorb a repair material. The ligament repair system further includes one or more suture assemblies configured to position the implant along or adjacent to a ruptured end of a torn ligament. The ligament repair system further includes a first fixation device configured to couple the one or more suture assemblies to a first bone. The ligament repair system further includes an allograft configured to be placed adjacent to the implant. The ligament repair system further includes at least one graft suture configured to position the graft along or adjacent to the implant or the ruptured end of the torn ligament. The ligament repair system further includes a second fixation device configured to couple the at least one graft suture to the first bone.

A further embodiment of the present disclosure includes a ligament repair system. The ligament repair system includes an implant sized and shaped for placement in synovial fluid, wherein the implant is configured to absorb a repair material. The ligament repair system further includes one or more suture assemblies configured to position the implant along or adjacent to a ruptured end of a torn ligament. The ligament repair system further includes a first fixation device configured to couple the one or more suture assemblies to a first bone. The ligament repair system further includes a synthetic graft configured to be placed adjacent to the implant. The ligament repair system further includes at least one graft suture configured to position the graft along or adjacent to the implant or the ruptured end of the torn ligament. The ligament repair system further includes a second fixation device configured to couple the at least one graft suture to the first bone.

A further embodiment of the present disclosure includes a tissue repair system. The tissue repair system includes a repair device including a graft sized and shaped for placement in a repair site of a torn tissue, and an implant wrapped around the graft, the implant being compressible and expandable and configured to absorb a repair material. The tissue repair system further includes at least one implant suture configured to position the implant along or adjacent to a ruptured end of the torn tissue. The tissue repair system further includes a first fixation device configured to couple the at least one implant suture to a first bone. The tissue repair system further includes at least one graft suture configured to position the graft along or adjacent to the implant or the ruptured end of the torn ligament. The tissue repair system further includes a second fixation device configured to couple the at least one graft suture to the first bone.

A further embodiment of the present disclosure includes a method for repairing a ruptured tissue. The method includes threading a first suture through a graft. The method further includes wrapping a scaffold around the graft. The method further includes positioning the scaffold and the graft proximate a ruptured end of the torn tissue of a patient. The method further includes securing the scaffold to a first bone with a first fixation device via the first suture.

### BRIEF DESCRIPTION OF THE DRAWINGS

The figures are illustrative only and are not required for enablement of the invention disclosed herein.

FIG. 1A is a diagrammatic representation of a torn anterior cruciate ligament;

FIG. 1B is a diagrammatic representation of a repair device having a fixation device and attached sutures;

FIG. 2A is a diagrammatic representation of the arthroscopic repair system according to an embodiment of the present disclosure.

FIG. 2B is a diagrammatic representation of the scaffold shown in FIG. 2A encasing the graft;

FIG. 3A is a diagrammatic representation of the arthroscopic repair system according to an embodiment of the present disclosure;

FIG. 3B is a diagrammatic representation of the scaffold shown in FIG. 3A wrapped around the graft;

FIG. 4 is a diagrammatic representation of the arthroscopic repair system according to an embodiment of the present disclosure;

FIG. 5A is a schematic showing the ruptured ligament shown in FIGS. 1A-3B;

FIG. 5B is a schematic showing the repair device inserted into the repair site using the arthroscopic repair system shown in FIG. 4;

FIG. 5C is a schematic showing the sutures, the fixation device, and the repair device shown in FIGS. 1A-3B being secured into the repair site;

FIG. 6A is a schematic depicting the repair system being utilized for ligament repair in the ACL;

FIG. 6B is a schematic depicting the repair device shown in FIG. 6A being inserted into a repair site;

FIG. 6C is a schematic depicting the repair system shown in FIGS. 6A-6B being fully inserted in the repair site; and

FIG. 6D is a schematic depicting the repair system shown in FIGS. 6A-6C being secured in the repair site.

### DETAILED DESCRIPTION OF THE INVENTION

Aspects of the invention relate to systems and methods for repairing a ruptured ligaments, such as an anterior cruciate ligament (“ACL”). The systems and methods described herein could be used for other ligaments as needed, such as the Achilles or other similar tissue. The system includes a combination of a scaffold and graft configured for the repair of the ruptured ligament, a fixation device, an one or more suture assemblies. The combination scaffold and graft allows the subject's body to develop a network of capillaries, arteries, and veins while provide improved mechanical stability. Well-vascularized connective tissues heal as a result of migration of fibroblasts into the scaffold. The methods and systems of the present disclosure provides a connection between the ruptured ligament, or forms around the torn ligament, and promotes the repair of the ruptured or torn ligament while maintaining the integrity and structure of the ligament.

The present disclosure provides a three-dimensional (3-D) scaffold encasing or wrapping around a graft for repairing a ruptured or torn ligament, such as an ACL. The scaffold and graft provide a connection between the ruptured ends of the ligament and fibers, or forms around the torn ligament, after injury, and encourages the migration of appropriate healing cells to form scar and new tissue in the scaffold. The scaffold is a bioengineered substitute for a fibrin clot and is implanted with a graft, for example, between the ruptured ends of the ligament fascicles, or wrapped around or placed adjacent to the torn ligament. This substitute scaffold and graft is designed to stimulate cell proliferation and extracellular matrix production in the gap between the ruptured ends of the ligament or the tear in the ligament, thus facilitating healing and regeneration.

As used herein, the injury may be a torn ligament or a ruptured ligament. A torn ligament may be a partial tear. A torn ligament may also refer to a complete tear. A partial tear is one where a portion of the ligament is damaged, but the ligament remains connected. The tear may be of any length or shape. A ruptured ligament, also known as a complete tear, is one where the ligament has been completely severed providing two separate ends of the ligament. A ruptured ligament may provide two ligament ends of similar or different lengths. The rupture may be such that a ligament stump is formed at one end. For example, there may be a tibial stump connected to the tibia and a femoral stump connected to the femur.

### Example ACL and Anatomy

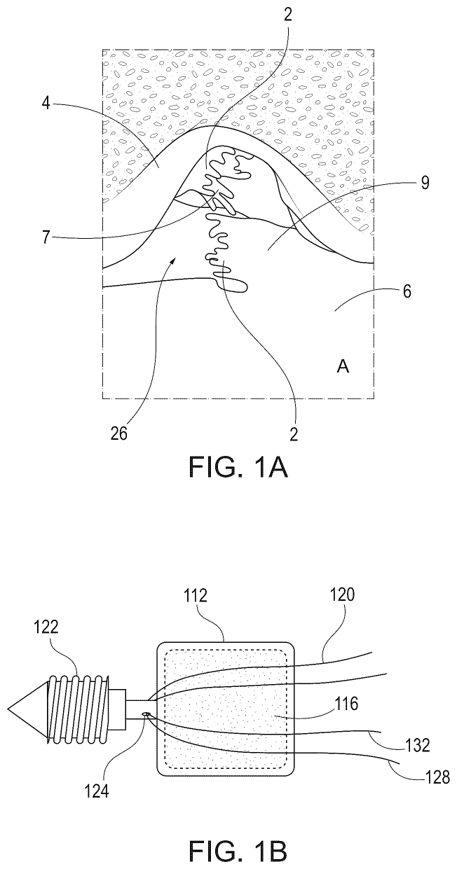

An example of a ruptured anterior cruciate ligament is depicted in FIG. 1A. The anterior cruciate ligament (ACL) 2 is one of four strong ligaments that connects the bones of the knee joint. The function of the ACL is to provide stability to the knee and minimize stress across the knee joint. It restrains excessive forward movement of the lower leg bone, the tibia 6, in relation to the thigh bone, the femur 4, and limits the rotational movements of the knee.

As shown in FIG. 1A, the anterior cruciate ligament 2 is ruptured such that it no longer forms a connection between the femur bone 4 and the tibia bone 6. The resulting ends of the ruptured ACL 2 may be of any length. The ends may be of a similar length, or one end may be longer in length than the other. The end on the femur 4 includes the femoral ACL stump 7. The end on the tibia 6 includes a tibial stump 9. In some instances, it is believed that a repair is desirable when the tibial stump length SL is less than about 75% of the effective ligament length LL but greater than 5% of a total length LL of the ACL. The total length of the ACL is considered to be the length of ligament from femoral footprint to the tibial footprint along a linear axis.

The knee joint includes tibial spines on the tibia 6 and the intercondylar notch of the femur 4. In some instances, the methods as described herein may include performing a notchplasty of the intercondylar notch of the femur to provide space for larger ligament to form after surgical repair using a scaffold. Such a notchplasty improves the size of the healing ligament, specifically resulting in a larger cross-sectional area of the ligament. As the mechanical strength of a ligament, and subsequently its ability to maintain the distance between the femur and tibia, is directly correlated with its cross sectional area, enlarging the notch with a notchplasty can help make a stronger repaired ACL and has been found by the inventors to be beneficial in ACL repair using a scaffold as described in the present disclosure.

### Scaffold

A scaffold of the present disclosure can be any shape that is useful for implantation into a subject. The scaffold, for instance, can be tubular, semi-tubular, cylindrical, including either a solid cylinder or a cylinder having hollow cavities, a tube, a flat sheet rolled into a tube so as to define a hollow cavity, liquid, an amorphous shape which conforms to that of the repair space, a “Chinese finger trap” design, a trough shape, or square. Other shapes suitable for the scaffold of the device as known to those of ordinary skill in the art are also contemplated in the invention.

Referring to FIG. 1B, the arthroscopic repair system of the present disclosure includes a repair device including a scaffold 112 and a graft 116. The system may further include arthroscopic equipment (not depicted). The present disclosure includes a scaffold 112, such that the scaffold 112 is configured for repair. The scaffold 112 is capable of being inserted into an area requiring repair and promotes regeneration of the ligament. The scaffold 112 is capable of insertion into a repair site and either forming a connection between the ends of the ruptured ligament, between bone, or forming around the torn ligament such that the integrity and structure of the ligament is maintained. Regeneration offers several advantages over reconstruction, previously used in ligament repair, including maintenance of the complex insertion sites and fan-shape of the ligament, and preservation of remaining proprioceptive fibers within the ligament substance.

For example, the scaffold 112 encases or wraps around a graft 116. The scaffold 112 may encase the graft 116 via a suture 120 or another mechanism. The scaffold 112 and graft 116 may be attached to a suture 120 and a fixation device 122. The fixation device 122 may, as shown in FIGS. 1B and 1C, be attached to the suture 120 through an eyelet 124 of the fixation device 122. In this configuration, the fixation device 122 is attached into a bone. The bone may be either the femur 4 or the tibia 6.

The scaffold 112 may function either as an insoluble or biodegradable regulator of cell function or simply as a delivery vehicle of a supporting structure for cell migration or synthesis. Numerous matrices made of either natural or synthetic components have been investigated for use in ligament repair and reconstruction. Natural matrices are made from processed or reconstituted tissue components (such as collagens and GAGs). Because natural matrices mimic the structures ordinarily responsible for the reciprocal interaction between cells and their environment, they act as cell regulators with minimal modification, giving the cells the ability to remodel an implanted material, which is a prerequisite for regeneration.

Synthetic matrices are made predominantly of polymeric materials. Synthetic matrices offer the advantage of a range of carefully defined chemical compositions and structural arrangements. Some synthetic matrices are not degradable. While the non-degradable matrices may aid in repair, non-degradable matrices are not replaced by remodeling and therefore cannot be used to fully regenerate ligament. It is also undesirable to leave foreign materials permanently in a joint due to the problems associated with the generation of wear particles, thus degradable materials are preferred for work in regeneration. Degradable synthetic scaffolds can be engineered to control the rate of degradation.

The scaffold 112 is preferably made of a compressible, resilient material which has some resistance to degradation by synovial fluid. Synovial fluid as part of normal joint activity, naturally prevents clot formation. This fibrinolytic process would result in the premature degradation of the scaffold and disrupt the healing process of the ligament. The material may be either permanent or biodegradable material, such as polymers and copolymers. The scaffold 112 can be composed, for example, of collagen fibers, collagen gel, foamed rubber, natural material, synthetic materials such as rubber, silicone and plastic, ground and compacted material, perforated material, or a compressible solid material.

The scaffold 112 may be a solid material such that its shape is maintained, or a semi-solid material capable of altering its shape and or size. The scaffold 112 may be made of expandable material allowing it to contract or expand as required. The material can be capable of absorbing plasma, blood, other body fluids, liquid, hydrogel, or other material the scaffold either comes into contact with or is added to the scaffold.

The scaffold material can be protein, lyophilized material, or any other suitable material. A protein can be synthetic, bioabsorbable or a naturally occurring protein. A protein includes, but is not limited to, fibrin, hyaluronic acid, elastin, extracellular matrix proteins, or collagen. The scaffold material may be plastic or self-assembling peptides. The scaffold material may incorporate therapeutic proteins including, but not limited to, hormones, cytokines, growth factors, clotting factors, anti-protease proteins (e.g., alpha1-antitrypsin), angiogenic proteins (e.g., vascular endothelial growth factor, fibroblast growth factors), antiangiogenic proteins (e.g., endostatin, angiostatin), and other proteins that are present in the blood, bone morphogenic proteins (BMPs), osteoinductive factor (IFO), fibronectin (FN), endothelial cell growth factor (ECGF), cementum attachment extracts (CAE), ketanserin, human growth hormone (HGH), animal growth hormones, epidermal growth factor (EGF), interleukin-1 (IL-1), human alpha thrombin, transforming growth factor (TGF-beta), insulin-like growth factor (IGF-1), platelet derived growth factors (PDGF), fibroblast growth factors (FGF, bFGF, etc.), and periodontal ligament chemotactic factor (PDLGF), for therapeutic purposes. A lyophilized material is one that is capable of swelling when liquid, gel or other fluid is added or comes into contact with it.

Many biological materials are available for making the scaffold, including collagen compositions (either collagen fiber or collagen gel), compositions containing glycosaminoglycan (GAG), hyaluronan compositions, and various synthetic compositions. Collagen-glycosaminoglycan (CG) copolymers have been used successfully in the regeneration of dermis and peripheral nerve. Porous natural polymers, fabricated as sponge-like and fibrous scaffolds, have been investigated as implants to facilitate regeneration of selected musculoskeletal tissues including ligaments. In one embodiment, the scaffold 112 is a sponge scaffold made from tendon (xenograft, allograft, autograft) or ligament or skin or other connective tissue which could be in the native state or processed to facilitate cell ingrowth or other biologic features.

In the illustrated embodiment the scaffold 112 is composed of a sponge or sponge-like material. The sponge scaffold 112 may be absorbable or nonabsorbable. The sponge scaffold 112 may include collagen, elastin, extracellular matrix protein, plastic, or self-assembling peptides. The sponge scaffold 112 may be hydrophillic. The sponge scaffold 112 is capable of compression and expansion as desired. For example, the sponge scaffold 112 may be compressed prior to or during implantation into a repair site. A compressed sponge scaffold allows for the sponge scaffold to expand within the repair site. The sponge may be lyophilized and/or compressed when placed in the repair site and expanded once in place. The expansion of the sponge scaffold 112 may occur after contact with blood or other fluid in the repair site or added to the repair site.

The sponge scaffold 112 may also be porous. The sponge scaffold 112 may be saturated or coated with a liquid, gel, or hydrogel repair material prior to implantation into a repair site. Coating or saturation of a sponge scaffold may ease implantation into a relatively undefined defect area as well as help to fill a particularly large defect area. The sponge scaffold 112 may be composed of collagen. In a preferred embodiment, the sponge scaffold 112 is treated with hydrogel. Examples of scaffolds and repair materials useful according to the invention are found in U.S. Pat. No. 6,964,685 and U.S. Patent Application Nos. 2004/0059416 and 2005/0261736, the entire contents of each are herein incorporated by reference.

An important subset of natural matrices are those made predominantly from collagen, the main structural component in ligament. Collagen can be of the soluble or the insoluble type. Preferably, the collagen is soluble, e.g., acidic or basic. For example, the collagen can be type I, II, III, IV, V, IX or X. Preferably the collagen is type I. More preferably the collagen is soluble type I collagen. Type I collagen is the predominant component of the extracellular matrix for the human ACL and provides an example of a choice for the basis of a bioengineered scaffold. Collagen occurs predominantly in a fibrous form, allowing design of materials with very different mechanical properties by altering the volume fraction, fiber orientation, and degree of cross-linking of the collagen. The biologic properties of cell infiltration rate and scaffold degradation may also be altered by varying the pore size, degree of cross-linking, and the use of additional proteins, such as glycosaminoglycans, growth factors, and cytokines. In addition, collagen-based biomaterials can be manufactured from a patient's own skin, thus minimizing the antigenicity of the implant (Ford et al., 105 Laryngoscope 944-948 (1995)).

### Fixation Device/Fixation

The present disclosure may also include one or more fixation devices 8. The fixation device 122 is a device capable of insertion into the bone such that it forms a stable attachment to the bone. In some instances, the fixation device 122 is capable of being removed from the bone if desired. The fixation device 122 may be conical shaped having a sharpened tip at one end and a body having a longitudinal axis. The body of the fixation device 122 may increase in diameter along its longitudinal axis. The body of the fixation device 122 may include grooves suitable for screwing the fixation device 122 into position. For example, as depicted in FIG. 1C, the fixation device 122 is screwed into the femur bone 4. The fixation device 122 may include an eyelet 124 at the base of the fixation device body through which one or more sutures may be passed. The eyelet 124 may be oval or round and may be of any size suitable to allow one or more sutures to pass through and be held within the eyelet 124.

The fixation device 122 may be attached to a bone by physical or mechanical methods as known to those of ordinary skill in the art. The fixation device 122 may also be attached directly to the graft 116. Alternatively, the fixation device 122 may be attached indirectly to the graft 116 using the suture 120 or the scaffold 112 to secure it in position. The fixation device 122 may have a central hole through which fluids, such as blood, may pass. The hole 24 may allow such fluids to flow onto the attached scaffold 112 and the graft 116.

The fixation device 122 includes, but is not limited to, a screw, a barb, an anchor, a helical anchor, a staple, a clip, a snap, a rivet, an endobutton, or a crimp-type anchor. The body of the fixation device 122 may be varied in length. Examples of fixation devices, include but are not limited to, IN-FAST™ Bone Screw System (Influence, Inc., San Francisco, CA), IN-TAC™ Bone Anchor System (Influence, Inc., San Francisco, CA), Model 3000 AXYALOOP™ Titanium Bone Anchor (Axya Medical Inc., Beverly, MA), OPUS MAGNUM® Anchor with Inserter (Opus Medical, Inc., San Juan Capistrano, CA), ANCHRON™, HEXALON™, TRINION™ (all available from Inion Inc., Oklahoma City, OK) and TwinFix AB absorbable suture anchor (Smith & Nephew, Inc., Andover, MA). Fixation devices are available commercially from manufacturers such as Influence, Inc., San Francisco, CA, Axya Medical Inc., Beverly, MA, Opus Medical, Inc., San Juan Capistrano, CA, Inion Inc., Oklahoma City, OK, and Smith & Nephew, Inc., Andover, MA.

The fixation device 122 may be composed of a non-degradable material, such as metal, for example titanium 316 LVM stainless steel, CoCrMo alloy, or Nitinol alloy, or plastic. The fixation device 122 is preferably bioabsorbable such that the subject is capable of breaking down the fixation device 122 and absorbing it. Examples of bioabsorbable material include, but are not limited to, MONOCRYL (poliglecaprone 25), PDS II (polydioxanone), surgical gut suture (SGS), gut, coated VICRYL (polyglactin 910, polyglactin 910 braided), human autograft tendon material, collagen fiber, POLYSORB, poly-L-lactic acid (PLLA), polylactic acid (PLA), polysulfone, polylactides (Pla), racemic form of polylactide (D,L-Pla), poly(L-lactide-co-D,L-lactide), 70/30 poly(L-lactide-co-D,L-lactide), polyglycolides (PGa), polyglycolic acid (PGA), polycaprolactone (PCL), polydioxanone (PDS), polyhydroxyacids, and resorbable plate material (see e.g. Orthopedics, October 2002, Vol. 25, No. 10/Supp.). The fixation device 122 may be bioabsorbed over a period of time which includes, but is not limited to, days, weeks, months or years.

In the illustrated embodiment, the fixation device 122 is attached to the scaffold 112 and the graft 116 using the suture 120. FIG. 1B illustrates an example of the fixation device 122 attached to the scaffold 112 and the graft 116 using the suture 120. The suture 120 is passed through the eyelet 124 of an fixation device 122 such that the fixation device 122 is attached to the scaffold 112 by the suture 120. The suture 120 has at least one free end. In some embodiments, a suture has two free ends, a first end 128 and a second end 132.

### Suture

In one embodiment, the suture 120 is bioabsorbable, such that the subject is capable of breaking down the suture and absorbing it, and synthetic such that the suture may not be from a natural source. In other embodiments, the suture 120 may be permanent such that the subject is not capable of breaking down the suture and the suture remains in the subject. The suture 120 may be rigid or stiff, or may be stretchy or flexible. The suture 120 may be round in shape and may have a flat cross section. Examples of sutures include, but are not limited to, VICRYL™ polyglactin 910, PANACRYL™ absorbable suture, ETHIBOND® EXCEL polyester suture, PDS® polydioxanone suture and PROLENE® polypropylene suture. Sutures are available commercially from manufacturers such as MITEK PRODUCTS division of ETHICON, INC. of Westwood, Mass.

In the illustrated embodiment, the suture 120 may be attached to one or both ends of a ruptured ligament 2 by its first end 128 and/or its second end 132. In one embodiment, the suture 120 may be passed through the eyelet 124 of the fixation device 122 and the first end 128 and second end 132 are tied to the ends of the distal ACL 2. The fixation device 122 is attached to the femur 4 by its sharpened end. The scaffold 112 and the graft 116 may be attached to the fixation device 122 by the suture 120 and held in position in the repair site 26. The fixation device 122 may be attached to either the tibia bone 6 or the femur bone 4 to secure the graft 116 and the scaffold 112 in position. In alternative embodiments, the scaffold 112 and the graft 116 may be attached to the femur bone 4 directly or indirectly. For example, the scaffold 112 may encase or be wrapped around the graft 116 via sutures or via another mechanism.

### Arthroscopic Equipment

Arthroscopic equipment may be configured to insert the suture 120 through the scaffold 112 and the graft 116. The arthroscopic equipment is further configured to position the scaffold 112 and the graft 116 between the ruptured end of the ligament 2 and the bone. The arthroscopic equipment may include an elongated delivery member. The elongated delivery member may include a channel that extends from a proximal end to a distal end of the elongated delivery member. The elongated delivery member may be sized and shaped to contain the scaffold 112 and the graft 116 attached to the suture 120 in the channel. At least a portion of the elongated delivery member may be further sized and shaped to be capable of being inserted into a repair site.

The arthroscopic equipment may be a syringe. The syringe may hold the suture 120 and the scaffold 112 encasing the graft 116 in place within the elongated delivery member of the syringe. The syringe may include a plunger configured to push the suture 120, the scaffold 112 and the graft 116 into a repair site such that the scaffold 112 and graft 116 are positioned along the suture 120 between the ruptured end of the ligament 2 and/or the bone. In alternative embodiments, the arthroscopic equipment may include a cannula, a container, and a pressure pump. In another embodiment, the arthroscopic equipment may further include a guiding suture that extends out of the distal end of the elongated delivery member. The guiding suture may be configured to pull and position the suture and the scaffold into the repair site.

### Repair Material

The scaffold 112 and the graft 116 can be pretreated with a repair material prior to implantation into a subject. The scaffold 112 and the graft 116 may be soaked in a repair material prior to or during implantation into the repair site 26. The repair material may be injected directly into the scaffold 112 prior to or during implantation. The repair material may be injected within a tubular scaffold at the time of repair. Repair material includes, but is not limited to, a gel, for example a hydrogel, a liquid, or collagen. A liquid includes any material capable of forming an aqueous material, a suspension or a solution. The repair material may include additional materials, such as growth factors, antibiotics, insoluble or soluble collagen (in fibrous, gel, sponge or bead form), a cross-linking agent, thrombin, stem cells, a genetically altered fibroblast, platelets, water, plasma, extracellular proteins and a cell media supplement. The additional repair materials may be added to affect cell proliferation, extracellular matrix production, consistency, inhibition of disease or infection, tonicity, cell nutrients until nutritional pathways are formed, and pH of the repair material. All or a portion of these additional materials may be mixed with the repair material before or during implantation, or alternatively, the additional materials may be implanted proximate to the defect area after the repair material is in place.

In certain embodiments, the repair material may include collagen and platelets. In some embodiments, platelets are derived from the subject to be treated. In other embodiments, platelets are derived from a donor that is allogeneic to the subject. In certain embodiments, platelets may be obtained as platelet rich plasma (PRP). In a non-limiting example, platelets may be isolated from a subject's blood using techniques known to those of ordinary skill in the art. As an example, a blood sample may be centrifuged at 700 rpm for 20 minutes and the platelet-rich plasma upper layer removed. Platelet density may be determined using a cell count as known to those of ordinary skill in the art. The platelet rich plasma may be mixed with collagen and used as a scaffold. The platelet rich plasma may be mixed with any one or more of the scaffold materials of the invention.

In one embodiment, the gel is a hydrogel. A hydrogel is a substance that is formed when an organic polymer (natural or synthetic) is crosslinked via covalent, ionic, or hydrogen bonds to create a three-dimensional open-lattice structure which entraps water molecules to form a gel. A polymer may be crosslinked to form a hydrogel either before or after implantation into a subject. For instance, a hydrogel may be formed in situ, for example, at a repair site. In certain embodiments, a polymer forms a hydrogel within the repair site upon contact with a crosslinking agent. Naturally occurring and synthetic hydrogel forming polymers, polymer mixtures and copolymers may be utilized as hydrogel precursors. See for example, U.S. Pat. No. 5,709,854. In certain embodiments, a hydrogel is a gel and begins setting immediately upon mixture and takes approximately 5 minutes to sufficiently set before closure of the defect and surgery area. Setting time may vary depending on the mixture of gel used and environmental factors.

For instance, certain polymers that can form ionic hydrogels which are malleable may be used to form the hydrogel. For example, a hydrogel can be produced by cross-linking the anionic salt of alginic acid, a carbohydrate polymer isolated from seaweed, with calcium cations, whose strength increases with either increasing concentrations of calcium ions or alginate. Modified alginate derivatives, for example, which have an improved ability to form hydrogels or which are derivatized with hydrophobic, water-labile chains, e.g., oligomers of ϵ-caprolactone, may be synthesized. Additionally, polysaccharides which gel by exposure to monovalent cations, including bacterial polysaccharides, such as gellan gum, and plant polysaccharides, such as carrageenans, may be crosslinked to form a hydrogel. Additional examples of materials which can be used to form a hydrogel include polyphosphazines and polyacrylates, which are crosslinked ionically, or block copolymers such as PLURONICS™ (polyoxyalkylene ether) or TETRONICS™ (nonionic polymerized alkylene oxide), polyethylene oxide-polypropylene glycol block copolymers which are crosslinked by temperature or pH, respectively. Other materials include proteins such as fibrin, polymers such as polyvinylpyrrolidone, hyaluronic acid and collagen. Polymers such as polysaccharides that are very viscous liquids or are thixotropic and form a gel over time by the slow evolution of structure, are also useful.

In another embodiment, the gel is hyaluronic acid. Hyaluronic acid, which forms an injectable gel with a consistency like a hair gel, may be utilized. Modified hyaluronic acid derivatives are particularly useful. Hyaluronic acid is a linear polysaccharide. Many of its biological effects are a consequence of its ability to bind water, in that up to 500 ml of water may associate with 1 gram of hyaluronic acid. Esterification of hyaluronic acid with uncharged organic moieties reduces the aqueous solubility. Complete esterification with organic alcohols such as benzyl renders the hyaluronic acid derivatives virtually insoluble in water, these compounds then being soluble only in certain aprotic solvents. When films of hyaluronic acid are made, the films essentially are gels which hydrate and expand in the presence of water.

As used herein, the term “pharmaceutically acceptable” means a non-toxic material that does not interfere with the effectiveness of the biological activity of the scaffold material or repair material. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art. The term “carrier” denotes an organic or inorganic ingredient, natural or synthetic, with which the scaffold material is combined to facilitate the application. The components of the pharmaceutical compositions also are capable of being co-mingled with the device of the present invention, and with each other, in a manner such that there is no interaction which would substantially impair the desired pharmaceutical efficacy.

### Graft

In the illustrated embodiment, the graft 116 is an ACL graft but several types of grafts could be used. Several types of ACL grafts are available for use by the surgeon in ACL reconstruction. The graft 116 may be an autologous graft or an autograft that is harvested from the patient. For example, these grafts may includes patellar bone-tendon-bone grafts, or hamstring grafts. Alternatively, the graft 116 may include one or more xenografts, allografts, isografts, or synthetic polymer grafts. Allografts include ligamentous tissue harvested from cadavers and appropriately treated and disinfected, and preferably sterilized. Xenografts include harvested connective tissue from animal sources such as, for example, porcine tissue. Typically, the xenografts must be appropriately treated to eliminate or minimize an immune response. Synthetic grafts include grafts made from synthetic polymers such as polyurethane, polyethylene, polyester and other conventional biocompatible bioabsorbable or nonabsorbable polymers and composites, such as the scaffolds described herein. Material for synthetic grafts may include, but are not limited to Supramid®, Teflon® or Dacron®, Proplast®, carbon fiber graft, ABC graft, Kennedy-LAD®, Trevia, Leeds-Keio, Gore-Tex®, PDS®, EULIT®, and Polyflex® or LARS®.

The graft 116 has a length L that extends along a longitudinal direction 2 and a width W that extends along a lateral direction 4. The dimensions of the graft 116 can be defined during manufacturing to any particular size. In another embodiment, the graft 116 may be positioned along or adjacent to the scaffold 112.

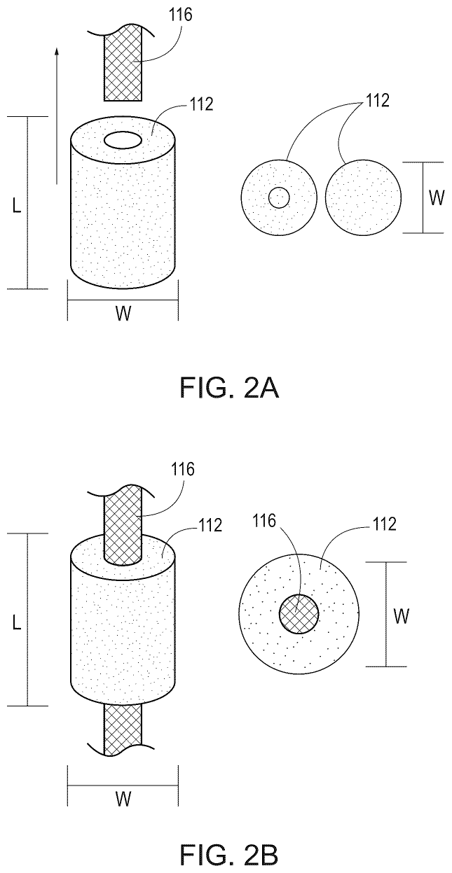

Referring to FIGS. 2A-2B, the scaffold 112 is a three-dimensional body configured to encase the graft 116. In the illustrated embodiment, the scaffold 112 is a cylindrical in shape that is hollow or tubular with one or more openings. The openings may be straight, curved, bent, looped, helical, zigzagged or any other shape. The graft 116 may be inserted and slid through the scaffold 112 in a straight, curved, helical or weaved path in and out of the scaffold. The scaffold 112 can therefore have one or more openings on either surface of the scaffold 112.

In alternative embodiments, the scaffold 112 may have any shape or cross section and be solid throughout the scaffold 112 or through a portion of the scaffold 112. In this configuration, the graft 116 may be threaded through the scaffold 112, displacing or removing (i.e. coring) the scaffold 112. Threading through a solid scaffold can displace or push aside the scaffold material while keeping the mass of the scaffold the same.

The scaffold 112 may cover the entirety of the graft 116 or a portion of the graft 116. The scaffold 112 is in direct contact with the graft 116 in at least one portion of the graft 116. The thickness of the scaffold 112 covering the graft 116 may vary from area to area. For example, the scaffold 112 may have a first thickness at the top and the bottom portions of the graft 116, and a second, greater thickness in the middle portion of the graft 116. The thickness of the scaffold 112 in any area may be 0.00 mm, creating a hole or window in the scaffold (for example, in a mesh-like scaffold). In one embodiment, the scaffold 112 covers the graft 116 such that the scaffold 112 completely wraps around the graft 116 in a 360 degree orientation around the longitudinal axis and without any gaps. In another embodiment, the scaffold 112 wraps around the graft 116 in a 360 degree orientation around the longitudinal axis with one or more gaps between the scaffold 112 and the graft 116.

In another embodiment, the graft 116 is threaded through the scaffold 112. The scaffold 112 may not have any seams and may not require any manipulation or preparation. Threading of the graft 116 may occur with or without a tool. The tool may act as a needle or guide for the graft 116 as it is threaded through the scaffold 112. Alternatively, the tool may act as a funnel for the scaffold 112.

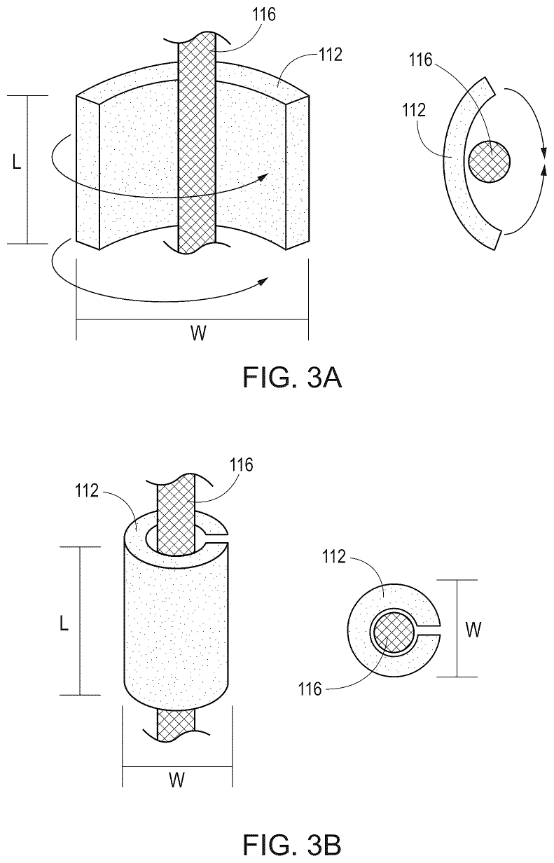

Referring to FIGS. 3A-3B, the scaffold 112 is a sheet of a desired thickness and geometry configured to be wrapped, folded, or clamped around the graft 116. The scaffold 112 may be a flat or pre-folded or pre-shaped sheet to fit around the graft 116. When the scaffold 112 is a flat sheet, the scaffold 112 will adjust its shape to the graft 116 to achieve the desired configuration. In the illustrated embodiment, the sheet is a rectangular sheet. In alternative embodiments, the sheet may be any other desired shape, including trapezoidal. The scaffold 112 may require manipulation to fit around the graft 116 and may include a visible seam corresponding to where the scaffold 112 was manipulated. The wrapping, folding, and/or clamping of the scaffold 112 around the graft 116 allows the scaffold 112 to envelop parts of the graft length-wise. This configuration results in one or more layers of the scaffold 112 around the graft 116 to achieve a desired overall thickness of scaffold material.

In one embodiment, the scaffold sheet 14 may be helically wrapped around the graft 116 such that the sheet has a greater length than width and requires multiple wraps around the graft 116. This configuration allows uncovered graft between the steps of the helix. In other embodiments, the sheet can be wrapped with a single wrap around the graft 116.

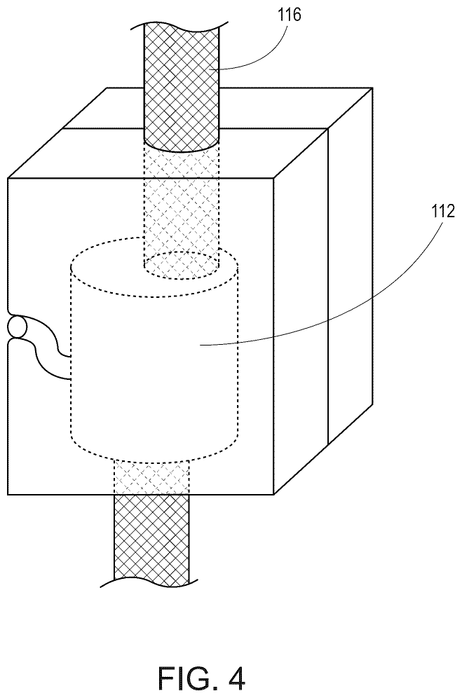

Referring to FIG. 4, the scaffold 112 may be formed directly around the graft 116 via various manual or automated casting, molding, or shaping methods. In the illustrated embodiment, the scaffold 112 is formed directly around the graft 116 via a die casting. In alternative embodiments, the scaffold 112 is formed around the graft 116 via molding cast. The graft 116 is placed in a die or mold which is closed around the graft 116. Scaffold material may then be inserted or injected into the die or mold, forming the scaffold 112 in the desired geometry. The scaffold material may be loose or particularized to be molded or shaped around the around the graft 116. The scaffold material is then combined to form the desired scaffold and casted, molded, or shaped around the graft 116 for the desired final configuration having a desired thickness or profile. The process of casting, molding, and/or shaping may be chemical, mechanical or electrical.

### Methods

Referring to FIGS. 1-4, aspects of the invention relate to methods of repairing a ruptured or torn ligament. In some embodiments, the scaffold 112, the graft 116, and the suture 120 is inserted into a repair site 26 of the ruptured or torn ligament 2 via arthroscopic equipment. In certain embodiments, a hole is drilled into a bone at or near a repair site of the ruptured or torn ligament 2 and the suture 120 is attached through the hole to the bone.

The repair site 26 is the area around a ruptured or torn ligament 2 into which a device may be inserted. The scaffold 112 and the graft 116 may be inserted into the repair site 26 during surgery via arthroscopic equipment using techniques known to those of ordinary skill in the art. The scaffold 112 is expandable and can either fill the repair site 26 or partially fill the repair site 26 while encasing or wrapping around the graft 116. The scaffold 112 can partially fill the repair site 26 when inserted and expand to fill the repair site 26 in the presence of blood, plasma or other fluids either present within or added into the repair site 26.

In one embodiment, the graft 116 may be coupled to one or more sutures and anchors to form a graft construct with desirable dimensions. The graft 116 may be encased in the scaffold 112 and advanced into the tibia and coupled to an anchor on the tibia. The graft 116 and/or the scaffold 112 may be further coupled to the femur 4 via an anchor and tensioned with the leg in extension.

In another embodiment, the scaffold 112 and the graft 116 may be attached directly or indirectly to the femur 4 and may contact the ruptured ligament 2. In another embodiment, the scaffold 112 and the graft 116 may form around the ruptured or torn ligament 2 at the repair site 26. For example, in one embodiment, the scaffold 112 and the graft 116 may be formed into a tube shape and wrapped around the ligament 2. In another embodiment, the scaffold 112 and the graft 116 may be positioned behind the ligament such that the ligament is held within the scaffold 112 and the graft 116. In yet another embodiment, the scaffold 112 and the graft 116 may be a “Chinese finger trap” design where one end is placed over a stump of a ruptured ligament and the second end placed over the other end of the ruptured ligament. In another embodiment, the graft may be positioned along or adjacent to the scaffold 112.

Aspects of the invention provide methods of repairing the ruptured ligament 2 involving drilling a hole at or near the repair site 26 of the ruptured ligament 2. A bone at or near a repair site is one that is within close proximity to the repair site and can be utilized using the methods and devices of the invention. For example, a bone at or near a repair site of a torn anterior cruciate ligament is a femur 4 bone and/or a tibia 6 bone. The hole can be drilled into a bone using a device such as a Kirschner wire (for example a small Kirschner wire) and drill, or microfracture pics or awls. One or more holes may be drilled into a bone surrounding the repair site 26 to promote bleeding into the repair site 26. The repair can be supplemented by drilling holes into the surrounding bone to cause bleeding. Encouraging bleeding into the repair site may promote the formation of blood clots and enhance the healing process of the injury.

The hole may be drilled into the femur 4 on the opposite side to the repair site 26. The scaffold 112, the graft 116, and the suture 120 may be inserted into the repair site 26 via arthroscopic equipment. The suture 120 may be passed through the hole in the bone and attached to the bone. The ruptured ligament 2 provides two ends of the ligament that were previously connected. In one embodiment, the scaffold 112 and the graft 116 may be attached to one or both ends 128, 132 of the ruptured ligament 2 by the suture 120. In another embodiment, the scaffold 112 and the graft 116 may be attached to one or both ends of the femur 4 and the tibia 6. The suture 120 may be attached to a second bone site at or near the repair site 26. In another embodiment, the hole may be drilled into the opposite side of the femur bone 4. The suture 120 is attached to the opposite side of the femur bone 4 using the first end 128 and the second end 132 through the hole 20 via arthroscopic equipment. In yet another embodiment, the hole is drilled into the tibia 6 near the end of the ruptured ligament 2 and the suture 120 is attached to the tibia 6 through the hole via arthroscopic equipment.

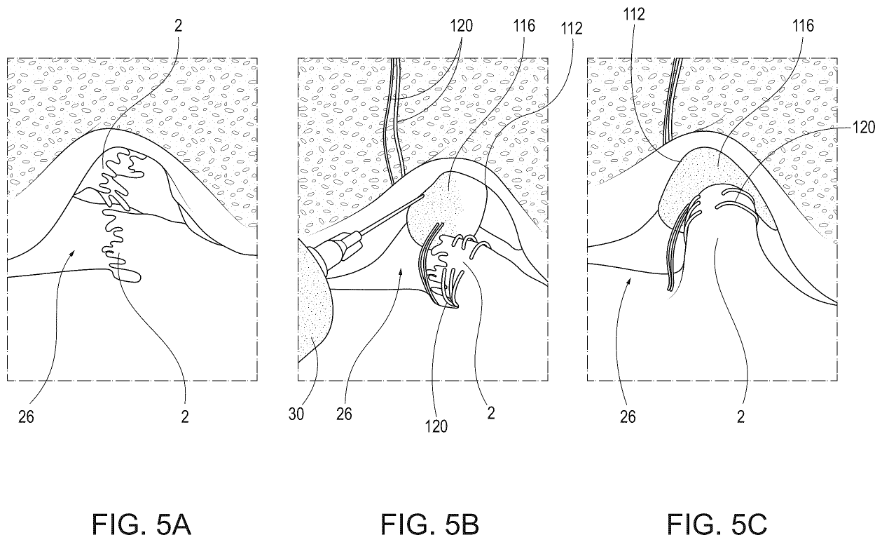

Now referring to FIGS. 5A-5C, the present disclosure includes an example of a surgical procedure which may be performed using the systems and methods disclosed. Prior to insertion of the scaffold 112 surrounding the graft 116, the affected extremity is prepared and draped in the standard sterile fashion. A tourniquet may be used if indicated. In FIG. 5A, after diagnostic arthroscopy is performed, the ruptured ligament 2 is identified and defined, the tissue ends are pretreated, either mechanically or chemically. The suture 120 is connected to a fixation device.

In FIG. 5B, prior to entering the repair site, arthroscopic equipment 30 attaches the suture 120 to the scaffold 112 surrounding the graft 116. The scaffold 112 and the graft 116 may be treated with a repair material. In one embodiment, the scaffold 112 and the graft 116 may also be pre-treated in antibiotic solution prior to implantation. The arthroscopic equipment 30 is configured to contain the scaffold 112, the graft 116, and the suture 120. During implantation, the arthroscopic equipment 30 introduces the scaffold 112, the graft 116, and the suture 120 into the tissue defect. In the illustrated embodiment, the arthroscopic equipment 30 introduces the repair device by pushing or releasing the repair device from the container into the repair site.

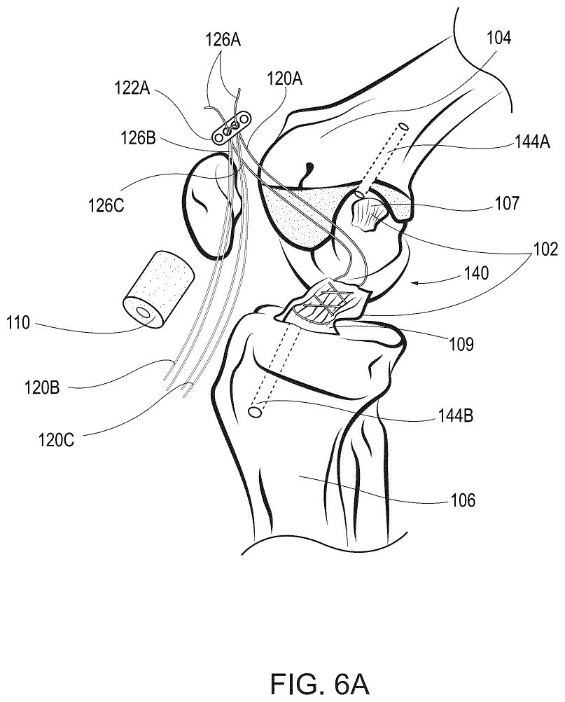

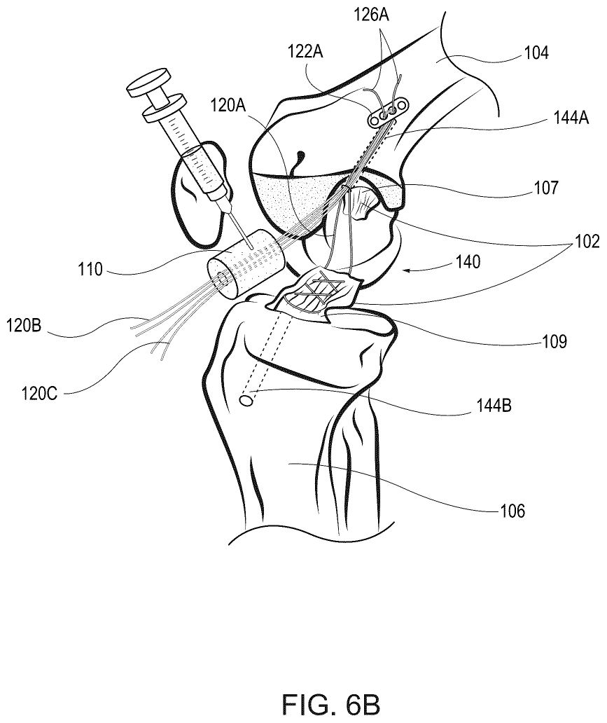

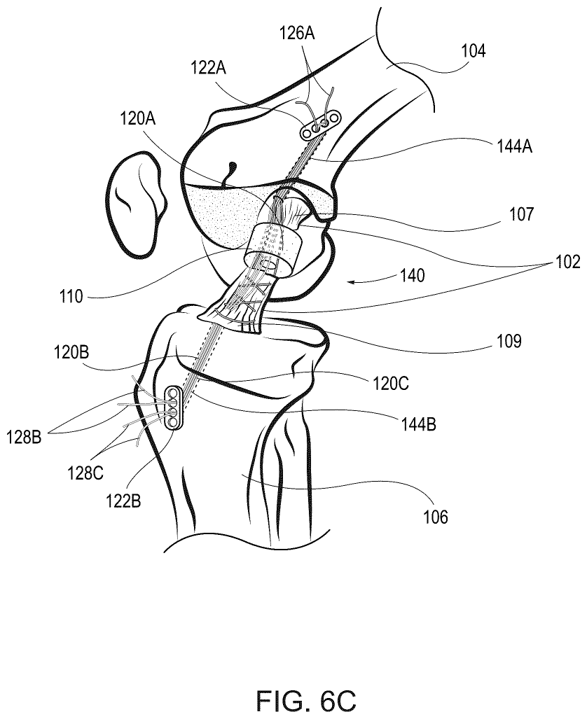

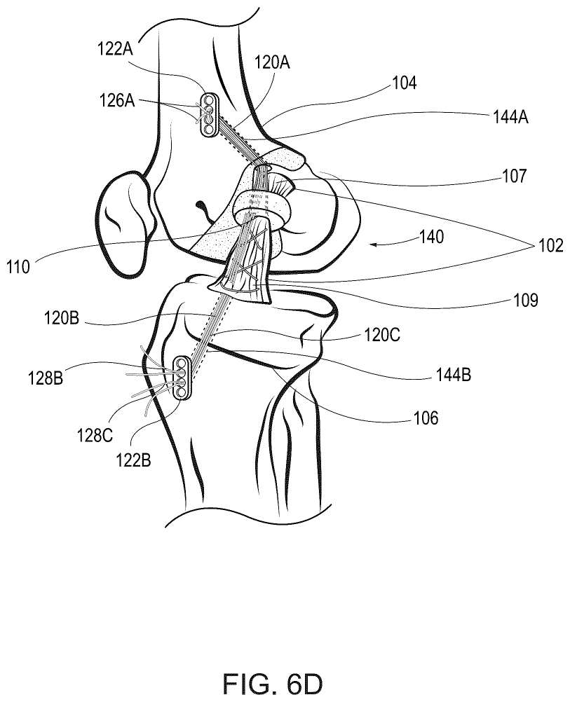

In the illustrated embodiment, the suture 120 is then connected to the ruptured end of the ligament 2 at the first end 128. In one embodiment, the suture 120 is placed through the ruptured end of the ligament 2 using a whip-stitch. In FIG. 6B, the fixation device 122 is passed through a bone, carrying suture 120. The fixation device 122 and the suture 120 is attached to the bone.

In FIG. 5C, the arthroscopic equipment 30 (not depicted) positions the scaffold 112 and the graft 116 along the suture between the ruptured ends of the ligament 2. In alternative embodiments, the arthroscopic equipment 30 positions the scaffold 112 and the graft 116 directly or indirectly onto the femur 4 and/or the tibia 6. The present disclosure may be used by insertion through an open incision. The scaffold 112 and the graft 116 is compressible to allow introduction through arthroscopic portals, incisions and equipment.

The scaffold 112 and the graft 116 is then bonded to the surrounding tissue using the methods described herein. This can be done by the addition of a chemical agent or a physical agent such ultraviolet light, a laser, or heat. The scaffold 112 and the graft 116 may be reinforced by placement of additional sutures or clips. The arthroscopic portals is closed, and a sterile dressing placed. The post-operative rehabilitation is dependent on the type and size of lesion treated, and the tissue involved.

Referring to FIGS. 6A-6D, aspects of the invention relate to methods of repairing a ruptured or torn ligament, such as an ACL, at a repair site 140. In some embodiments, the scaffold 112, the graft 116, and one or more sutures 120 are inserted into the repair site 140 of the ruptured or torn ligament 102 via arthroscopic equipment 130.

The repair site 140 is the area around a ruptured or torn ligament 102 into which a device may be inserted. The scaffold 112 and the graft 116 may be inserted into the repair site 140 during surgery via the arthroscopic equipment 130. The scaffold 112 is expandable and can either fill the repair site 140 with the graft 116 or partially fill the repair site 140 with the graft 116. The scaffold 112 can partially fill the repair site 140 when inserted and expand to fill the repair site 140 in the presence of blood, plasma or other fluids either present within or added into the repair site 140.

In the illustrated embodiment, the scaffold 112 and the graft 116 are coupled to form the repair device 110 and the repair device 110 is attached directly or indirectly to bone and contacts the ruptured or torn ligament 102. In another embodiment, the repair device 110 may form around the ruptured or torn ligament 102 at the repair site 140. For example, in one embodiment, the repair device 110 is wrapped around the ligament 102, in another embodiment, the repair device 110 is positioned behind the ligament such that the ligament is held within the repair device 110. In yet another embodiment, the repair device 110 may be a “Chinese finger trap” design where one end is placed over a stump of a ruptured ligament and the second end is placed over the other end of the ruptured ligament.

In an alternative embodiment, the graft 116 is coupled to the one or more sutures 120 and one or more fixation devices 122 to form a graft construct with desirable dimensions. The graft 116 is advanced into a first bone and coupled to the one or more fixation devices 122 on the first bone. The graft 116 is further coupled to a second bone via the one or more fixation devices 122 and tensioned in extension. The graft 116 is subsequently coupled to the scaffold 112.

An example of a ruptured anterior cruciate ligament is depicted in FIG. 6A. The anterior cruciate ligament (ACL) 102 is one of four strong ligaments that connects the bones of the knee joint. The function of the ACL is to provide stability to the knee and minimize stress across the knee joint. It restrains excessive forward movement of the lower leg bone, the tibia 106, in relation to the thigh bone, the femur 104, and limits the rotational movements of the knee.

As shown in FIGS. 6A-6D, the anterior cruciate ligament 102 is ruptured such that it no longer forms a connection between the femur bone 104 and the tibia bone 106. The end on the femur 104 includes the femoral ACL stump 107. The end on the tibia 106 includes a tibial stump 109. Aspects of the invention provide methods of repairing the ruptured ligament 102 involving drilling one or more holes 144 at or near the repair site 140 of the ruptured ligament 102. In FIG. 6A, holes 144A, 144B are drilled into the femur 104 and the tibia 106, respectively, at the repair site 140. The hole 144A may be additionally referred to hereinafter as the femoral tunnel 144A and the hole 144B may be additionally referred to hereinafter as the tibial tunnel 144B A first suture 120A is placed through the tibial stump 109 using a whip-stitch.

The first suture 120A is attached via the first end 126A to a first fixation device 122A. A second suture 120B and a third suture 120C are coupled to the fixation device 122A at respective first ends 126B, 126C. The fixation device 122A is subsequently passed through the femoral tunnel 144A and coupled to the femur 104. In FIG. 6B, the repair device 110 is loaded onto the second and third sutures 120B, 120C. The scaffold 112 is then injected with repair material as described. The repair device 110 and the second and third sutures 120 may be inserted into the repair site 140 via the arthroscopic equipment 130. In FIG. 6C, the free ends 128B, 128C of the second and third sutures 120B, 120C are passed through the tibial tunnel 144B and are coupled to a second fixation device 122B coupled to the tibia 106. The repair device 110 is then positioned between the two ends of the torn ACL 2. In FIG. 6D, the knee is extended and the sutures 120A, 120B, 120C, and the fixation devices 122A, 122B are secured.

In alternative embodiments, the scaffold 112 and the graft 116 may be attached to the femur bone 104 directly or indirectly. For example, the graft 116 may be attached to the scaffold 112 via sutures or via another mechanism. In another embodiment, the graft 116 may be positioned along or adjacent to the scaffold 112. In one example, the scaffold 112 may be attached between the femoral insertion point and the tibial insertion point and may sit conjunctive to the graft 116. In another example, the graft 116 may be inserted or pushed through the scaffold 112.

In the present disclosure, a subject includes, but is not limited to, any mammal, such as human, non-human primate, mouse, rat, dog, cat, horse or cow. In certain embodiments, a subject is a human. The present disclosure may also include kits for repair of ruptured or torn ligaments. A kit may include a scaffold of the invention having at least one fixation device attached to the scaffold and instructions for use. The scaffold may further include one or more sutures that attach an fixation device to the scaffold. A kit may further include a container that contains a repair material as described herein.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention. Those skilled in the art will recognize or be able to ascertain using no more than routine experimentation, many equivalents to the specific embodiments of the invention described herein. Such equivalents are intended to be encompassed by the following claims.

All references disclosed herein are incorporated by reference in their entirety.

What is claimed:

1. A ligament repair system, comprising:
a repair device configured to repair a ligament and for placement in synovial fluid, the repair device having a hydrophilic scaffold covering a graft;
one or more suture assemblies configured to position the repair device along or adjacent to a ruptured end of a torn ligament; and
a fixation device configured to couple the one or more suture assemblies to a bone.

2. The ligament repair system according to claim 1, wherein the hydrophilic scaffold is a collagen scaffold.

3. The ligament repair system according to claim 1, wherein the fixation device is configured for placement against the bone.

4. The ligament repair system according to claim 1, wherein the graft is an ACL graft.

5. The ligament repair system according to claim 4, wherein the graft is an autograft, an allograft, or a synthetic graft.

6. The ligament repair system according to claim 1, wherein the scaffold and one or more suture assemblies are preloaded and insertable through an arthroscopic cannula.

7. The ligament repair system according to claim 1, wherein the graft and the one or more suture assemblies are preloaded and insertable through an arthroscopic cannula.

8. The ligament repair system according to claim 1, wherein the scaffold and the graft and the one or more suture assemblies are preloaded and insertable through an arthroscopic cannula.

9. The ligament repair system of claim 1, wherein the ligament is an ACL and wherein the scaffold is configured to permit cell ingrowth.

10. The ligament repair system of claim 1, wherein the fixation device is selected from the group consisting of a screw, a barb, a helical fixation device, a staple, a clip, a snap, and a rivet.

11. The ligament repair system of claim 1, further comprising a repair material.

12. The ligament repair system of claim 11, where the repair material is a platelet or plasma.

13. The ligament repair system of claim 1, wherein the scaffold is in direct contact with the graft.

14. The ligament repair system of claim 1, wherein the scaffold covers the entirety of the graft.

15. The ligament repair system of claim 1, wherein the scaffold covers a portion of the graft.

16. The ligament repair system of claim 1, wherein the scaffold is wrapped helically around the graft.

17. The ligament repair system of claim 1, wherein the scaffold is wrapped around the graft.

18. The ligament repair system of claim 1, wherein the graft is threaded through the scaffold.

19. The ligament repair system of claim 18, wherein the scaffold is a hollow tubular shape with a central channel and one or more openings into the channel.

20. The ligament repair system of claim 18, wherein the graft displaces the scaffold material when threaded through the scaffold such that the mass of the scaffold does not change.

21. The ligament repair system of claim 18, wherein the graft replaces the scaffold material when threaded through the scaffold such that the mass of the scaffold changes.

22. The ligament repair system of claim 1, wherein the scaffold is wrapped, folded, or clamped around the graft.

23. The ligament repair system of claim 1, wherein the scaffold is initially pre-shaped to fit on or around the graft when combined with the graft such that the scaffold adjusts its shape to the graft.

24. The ligament repair system of claim 1, wherein the scaffold includes one or more layers surrounding the graft.

25. The ligament repair system of claim 1, wherein the scaffold is molded around the graft.

26. A method for repairing an anterior cruciate ligament, comprising:
a. inserting a repair device proximate a ruptured end of a torn ligament of a patient, the repair device comprising a combination of a scaffold and a graft;
b. securing the repair device to a suture and a first bone with a first fixation device;
c. coupling the repair device to a second bone with a second fixation device.

27. The method of claim 26, further comprising attaching the first fixation device only indirectly to the repair device.

28. The method of claim 26, further comprising attaching the first fixation device to the femur.

29. The method of claim 26, further comprising positioning the repair device between the ruptured end of the ligament and the femur.

30. The method of claim 26, wherein the scaffold comprises a porous collagen sponge.

31. The method of claim 26, further comprising treating the scaffold with a repair material.

32. The method of claim 31, wherein the repair material is a blood, platelet or plasma.

33. The method of claim 26, wherein the first fixation device is bioabsorbable.

34. The method of claim 26, wherein the suture is bioabsorbable.

35. The method of claim 26, wherein the first fixation device is an endobutton.

36. The method of claim 26, wherein the graft is an autologous graft.

37. The method of claim 36, further comprising:
harvesting a portion of a hamstring from the patient, and
forming the graft from the portion of the hamstring.

38. The method of claim 26, wherein the graft is an allograft.

39. The method of claim 38, further comprising:
harvesting a portion of a hamstring from another patient, and
forming the graft from the portion of the hamstring.

40. The method of claim 26, wherein the graft is a synthetic graft.

41. The method of claim 40, further comprising:
assembling a bundle of synthetic polymer having a longitudinal dimension into the graft.

42. A tissue repair system, comprising:
a repair device including:
a graft sized and shaped for placement in a repair site of a torn tissue, and
an implant wrapped around the graft, the implant being compressible and expandable and configured to absorb a repair material;
at least one implant suture configured to position the implant along or adjacent to a ruptured end of the torn tissue;
a first fixation device configured to couple the at least one implant suture to a first bone;
at least one graft suture configured to position the graft along or adjacent to the implant or the ruptured end of the torn ligament; and
a second fixation device configured to couple the at least one graft suture to the first bone.

43. A method for repairing a ruptured tissue, comprising:
threading a first suture through a graft;
wrapping a scaffold around the graft;
positioning the scaffold and the graft proximate a ruptured end of the torn tissue of a patient; and
securing the scaffold to a first bone with a first fixation device via the first suture.
